(19) United States
(12) Patent Application Publication
(10) Pub. No.: US 2013/0211524 A1
(43) Pub. Date: Aug. 15, 2013

(54) LAMINOPLASTY IMPLANT, IN PARTICULAR FOR CERVICAL LAMINOPLASTY

(71) Applicant: Pascal Mousselard Hugues, Paris (FR)

(72) Inventor: Pascal Mousselard Hugues, Paris (FR)

(73) Assignee: MEDICREA INTERNATIONAL, Neyron (FR)

(21) Appl. No.: 13/879,654

(22) PCT Filed: Oct. 19, 2011
(86) PCT No.: PCT/IB2011/054669

§ 371 Date: Apr. 16, 2013

(30) Foreign Application Priority Data
Oct. 28, 2010 (FR) 10 58911

(51) Int. Cl. A61F 2/44

(52) U.S. Cl. CPC A61F 2/44
USPC 623/17.11

(57) ABSTRACT

An implant (1) includes a body (5) and intermediate and side walls (15, 16, 17) forming anchoring parts able to be anchored on portions of lamina (101). The anchoring parts are “inverted U”-shaped, so that, when the body (5) is arranged in the implantation position, the direction of engagement of these walls on the portions of lamina (101) is parallel to the cranial-caudal direction of the vertebral column, i.e. the general longitudinal direction of the vertebral column, and the central body (5) includes mounting element (3) allowing one or more grafts (103) to be mounted thereon that can extend from one resected surface (102) of a portion of lamina (101) to the resected surface (102) of the other portion of lamina (101), so that the ends of that or those grafts (103) can be situated, after placement, in the immediate vicinity of the resected surfaces (102).

The present invention relates to a laminoplasty implant, in particular for cervical laminoplasty.

It is known to treat a compression of the spinal cord at the cervical vertebrae through a so-called “laminoplasty” operation, consisting of enlarging the vertebral canal.

One known laminoplasty technique consists of cutting a lateral zone of the laminae of one or more vertebrae, then producing outward pivoting of one or two portions of lamina defined by said cutting, and putting an implant in place between these portions of lamina in order to keep the latter in that pivoted position. An implant adapted to this technique is described by documents US 2004/030388 Al (FIGS. 4 to 7) or CN 101 785 694 A.

Another known laminoplasty technique, called “double door,” consists of performing an ablation of the central zone of the lamina, producing two lateral grooves at the base of the portions of lamina thus formed, on the outer side, both to reduce the thickness of the bone until its flexibility is allowed and to produce a recess making it possible to pivot each portion of lamina toward the outside, pivoting each portion of lamina toward the outside, and lastly placing an implant on the portions of lamina. One implant adapted to this technique is described by document CN 201 453 355 U or US 2003/045936 A1.

Still another known laminoplasty technique consists of performing an ablation of the central zone of the lamina, and then connecting the two portions of lamina thus formed using an implant. One known implant adapted to the latter technique is disclosed by documents US 2004/030388 A1 (FIGS. 20 to 22) or FR 2 918 262 A1. Such an implant comprises a central body in the form of a strip and lateral ends in the form of U clips, the bottom walls of said U clips being situated on the outer side of the implant and, when implanted, covering the resected surfaces of free ends of the portions of lamina.

An implant of this type has the drawback of not being very secure for the practitioner to place in light of the risk of lesion of the spinal cord during impacting of said clips on the inner and outer faces of the portions of lamina. Furthermore, these existing implants do not allow perfect reconstruction of the bone bridge naturally formed by the posterior arc of a vertebra, nor, as a result, complete reinsertion of the soft tissues normally anchored on a lamina or a spinous process.

The present invention aims to resolve these drawbacks. The implant it concerns is adapted to connect the two portions of the lamina of a vertebra resulting from an ablation of the central zone of said lamina, and to that end comprises, in a known manner:
a body having a first end intended to be connected to a first portion of lamina and a second end intended to be connected to the second portion of lamina, said body having, in its implantation position, an outer side, an inner side, an upper side and a lower side; a first direction extends from the outer side toward the inner side of said body; and
two anchoring portions to said portions of lamina, one of which is connected to said first end and the other of which is connected to said second end, each anchoring portion being in the shape of a U, i.e. comprising an inner side wall intended to come against an inner side of the corresponding portion of lamina, an outer side wall intended to come against an outer side of said portion of lamina, and an intermediate wall connecting said inner side wall to said outer side wall; a second direction extends from said intermediate wall, substantially perpendicularly thereto, toward the opening of the U defined between the free end edges of said inner side wall and said outer side wall.

According to the invention,
said intermediate wall of each anchor portion is situated toward the upper side of the body of the implant, and said second direction is substantially perpendicular to said first direction, said inner, outer and intermediate side walls thus being arranged relative to said body so that, when the body is arranged in said implantation position, the direction of engagement of these walls on the portions of lamina is parallel to the cranial-caudal direction of the vertebral column, i.e. the general longitudinal direction of said vertebral column; and
said central body comprises mounting means allowing one or more grafts to be mounted thereon so as to extend from one resected surface of a portion of lamina to the resected surface of the other portion of lamina, the ends of that or those grafts being thus situated, after placement, in the immediate vicinity of said resected surfaces.

Thus, the implant according to the invention comprises side and intermediate walls whereof the engagement on the portions of lamina is done in the cranial-caudal direction, i.e. parallel to the general longitudinal direction of the vertebral column. This engagement is done using a much safer process, in terms of risks of lesion of the spinal cord, than the engagement of an implant according to the prior art, done in the antero-posterior direction, i.e. perpendicular to the general longitudinal direction of the vertebral column. Moreover and above all, this structure makes it possible to leave the resected surfaces of the portions of lamina free and therefore makes it possible to place one or more grafts extending from one portion of lamina to the other as far as the vicinity of said resected surfaces. The reconstruction of a bone bridge between the portions of lamina can then be done under better conditions, by growth of the bone cells of the portions of lamina toward the graft(s). This bone bridge makes it possible to perform a perfect reinsertion of the soft tissues normally anchored on a lamina or a spinous process.

Furthermore, said intermediate walls, owing to their bearing against the portions of lamina, constitute stop surfaces making it possible to completely stabilize the implant in said implantation position.

Advantageously, each intermediate wall is deformable between a first position, in which the outer side wall to which said intermediate wall is connected is remote from the inner side wall to which said intermediate wall is connected, and a second position, in which said outer side wall is closer to said inner side wall so as to grip a portion of lamina between it and said inner side wall.

Said first position is an insertion position of the implant, in which the outer side wall is moved away from the corresponding inner side wall, which greatly facilitates the insertion of the implant on a vertebra. Once said implantation position is reached, the intermediate walls are deformed so as to fold the outer side walls down against the portions of lamina, in said second position, and to thereby grip the portions of lamina between said outer and inner walls, thereby ensuring fastening of the implant.

The implant according to the invention thus has still further increased safety relative to the risk of lesion of the patient's spinal cord during placement of said implant, no impaction being necessary to insert, more or less forcibly, the clips on said portions of lamina, as is the case for the implants according to the prior art. Preferably, each inner side wall extends in a direction forming, with the inner side of the body of the implant, an angle going from 100 to 120°. This angle is preferably 110°.

The two side walls, as well as the intermediate walls connected to them, thus diverge from one another in the direction moving away from said inner side of the body. This shape is completely adapted to the shape of the cut out, and potentially pivoted, portions of lamina.

Each side wall and/or each intermediate wall can comprise means favouring its fastening relative to a portion of lamina, in particular sharp teeth that can be inserted into the bone of said portion of lamina, and/or one or more holes making it possible to place screws through said portion of lamina.

Preferably, said mounting means comprised by said central body for assembling one or more grafts are in the form of a cage having a transverse conduit, the ends of which emerge laterally.

This transverse conduit allows easy placement of the graft(s) on the implant, and perfect maintenance of said graft(s).

Said case can form a single piece with said body, or can be formed by a piece separate from said body and fixed thereon. In the first case, the implant assembly can in particular be made from a biocompatible material, in particular titanium; in the second case, said body and said side and intermediate walls are made from a piece of titanium while said cage is made from a synthetic material, in particular PEEK (polyetheretherketone). The two-piece version offers more possibilities as to the shapes the cage can be given.

Said cage can assume a flat triangular shape, similar to that of a spinous process, and thus allow an at least approximate reconstitution of said spinous process when the latter is resected.

According to one preferred embodiment of the invention, in that case, said cage has concave lateral edges and rounded corners. It can also have, seen in a transverse direction, a height that gets smaller on the side opposite said body.

The invention will be well understood, and other features and advantages thereof will appear, in reference to the appended diagrammatic drawing, showing, as non-limiting examples, two embodiments of the concerned laminoplasty implant.

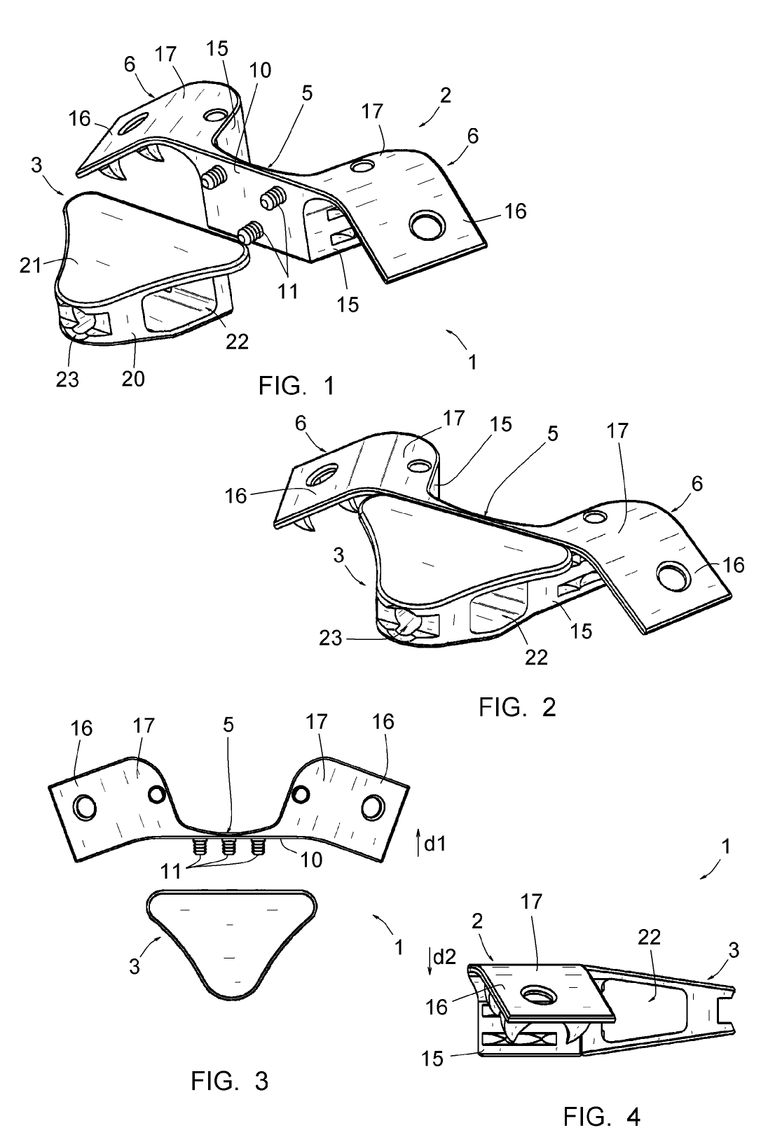

FIG. 1 is a perspective view, according to a first embodiment, before assembly;

FIG. 2 is a view similar to FIG. 1, after assembly;

FIG. 3 is a top view, before assembly;

FIG. 4 is a side view;

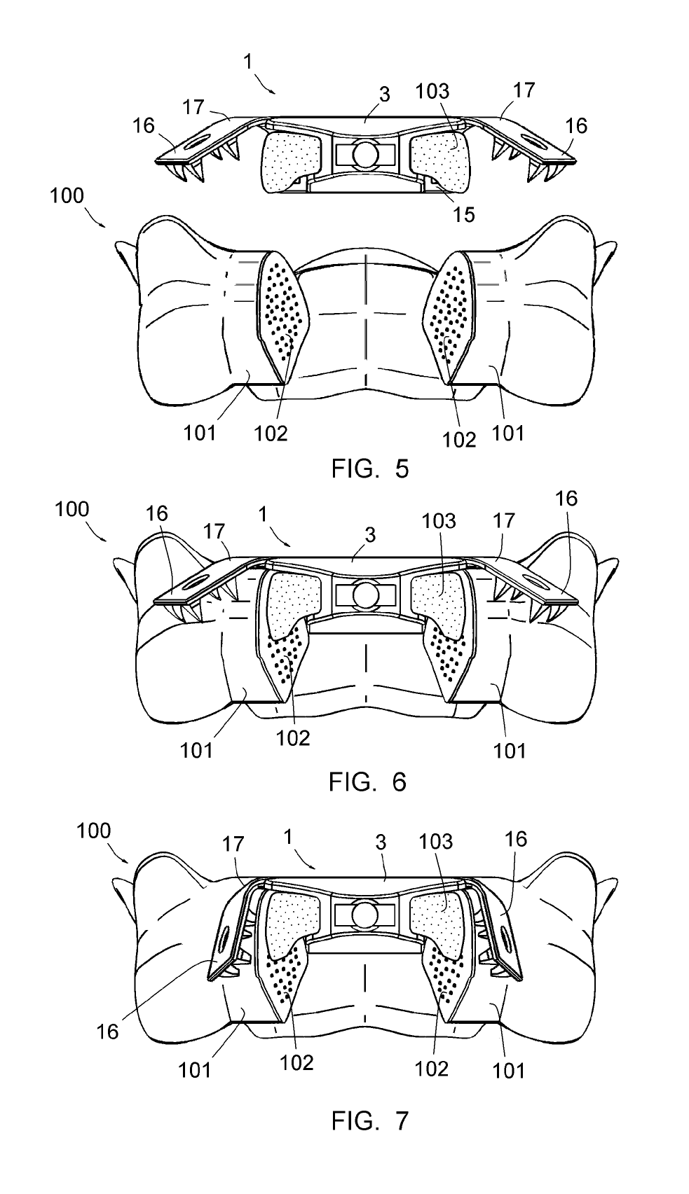

FIG. 5 is a front view, posterior side, before placement on a vertebra whereof the central portion of the lamina has been resected and the portions of lamina of which generated by said resection have been pivoted outwardly;

FIG. 6 is a view similar to FIG. 5, during placement on the vertebra;

FIG. 7 is a view similar to FIG. 6, during fastening on the vertebra;

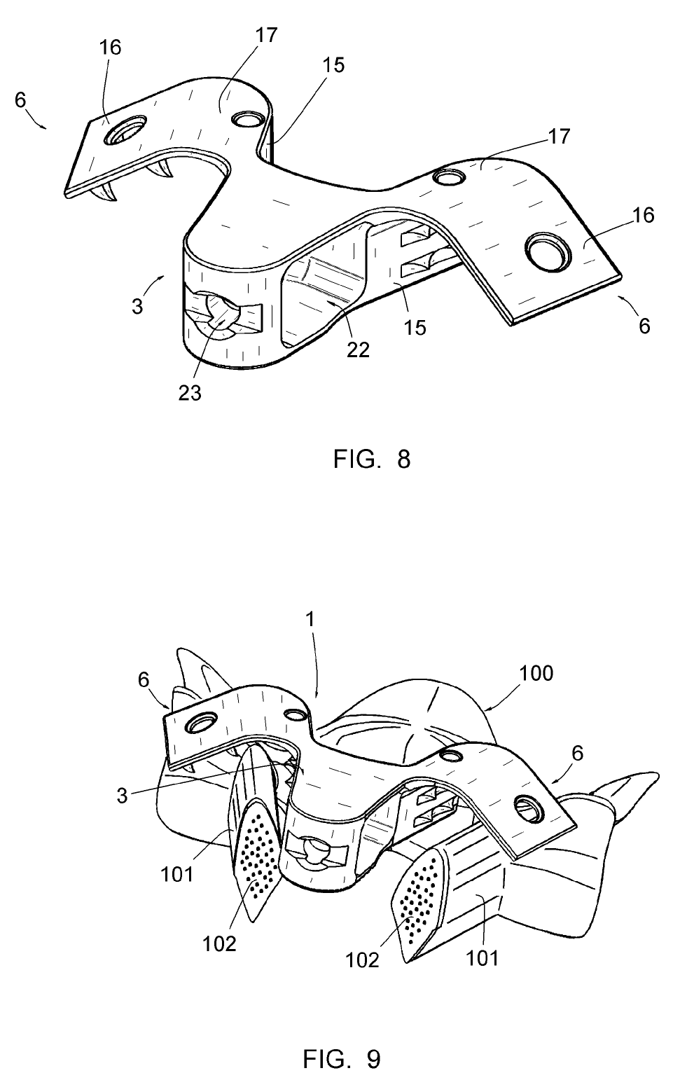

FIG. 8 is a perspective view, according to a second embodiment, and

FIG. 9 is a view similar to FIG. 8, before placement on a vertebra whereof the central portion of the lamina has been resected and the portions of lamina of which generated by said resection have been pivoted outwardly.

For simplification purposes, the elements or parts of the implant according to the first embodiment that are found identically or similarly on the implant according to the second embodiment will be designated using the same numerical references and will not be described again.

FIGS. 1 to 4 show a laminoplasty implant 1, in particular for cervical laminoplasty, that comprises a base piece 2 made from a biocompatible metal material, in particular titanium, and a cage 3 made from a synthetic material, in particular PEEK (polyetheretherketone).

The base piece 2 forms a body 5 and two laminar anchoring side portions 6. The body 5 is formed by a wall 10 that can, in an implantation position (see FIGS. 6 and 7), connect the two portions 101 of a cervical vertebral lamina resulting from an ablation of the central portion of said lamina. The inner face of said wall 10, intended to face the vertebral canal, is smooth, while the outer surface of said wall comprises two lugs 11 with sharp skirts, for assembling the cage 3. A direction d1 extending from the outer side to the inner side of the body 5 (see FIG. 3) is defined as a “first direction”.

Each laminar anchoring side portion 6 comprises an inner side wall 15, an outer side wall 16 and an intermediate wall 17 connecting the wall 15 to the wall 16 so that each anchoring portion 6 is in the shape of a U. A direction extending from the intermediate wall 17, substantially perpendicular thereto, toward the opening of the U defined between the free end edges of the side walls 15 and 16 (see FIG. 4) is defined as a “second direction”.

Each inner side wall 15 is adjacent to the corresponding end of the body 5, with which it forms a body, and includes sharp teeth protruding from its outer surface, i.e. its surface facing the corresponding outer side wall, in particular four teeth arranged in a square as in the illustrated example. As shown in FIG. 3, the side 15 and intermediate 17 walls extend in a direction forming, with the inner surface of the body 5, a substantially 110° angle.

Each outer side wall 16 is connected to the corresponding intermediate support wall 17. It is passed through by a hole for receiving a screw allowing it to be fixed to the portion of lamina 101 and includes sharp teeth, with a curved shape, protruding from its inner surface, i.e. its surface facing the corresponding inner side wall 15, in particular four teeth arranged in a square as in the illustrated example.

Each intermediate support wall 17 is adjacent, on two opposite edges, to the corresponding inner side wall 15 and outer side wall 16, respectively. It also comprises a hole for receiving a screw allowing it to be fixed to the portion of lamina 101. Each intermediate wall 17 is also deformable non-elastically between an insertion position of the implant, shown in FIGS. 1 to 6, in which each outer side wall 16 is moved away from the corresponding inner side wall 15, and a fixing position of the implant 1, shown in FIG. 7, in which said outer side wall 16 is brought closer to the corresponding inner side wall 15 so as to grip the portion of lamina 101 between it and said inner side wall 15, with penetration of the teeth of said side walls into the bone of the portion of lamina 101.

In particular, in the illustrated example, the wall 16 and the wall 17 of each anchoring portion are formed by a same flexible material, the wall 16 being in the continuous extension of the wall 17.

As shown clearly in FIGS. 3 and 4, said second direction d2 is substantially perpendicular to said first direction d1.

The cage 3 has a flat triangular shape, similar to that of a spinous process, and at a height that gets smaller on the side opposite said body 5 (see FIG. 4). It comprises a body 20 and an upper wall 21 with a triangular shape, with concave side edges and rounded corners. The body 20 forms a transverse conduit 22 whereof the ends emerge laterally, and comprises an anterior wall pierced with holes for forcibly receiving lugs 11 and a posterior wall having a cavity 23 for receiving a handling instrument.

As understood in reference to FIGS. 1 and 2, the implant 1 is formed by assembling the cage 3 to the base piece 2, said assembly being done by forcible insertion of the lugs into the holes comprised by the anterior wall of the cage 3.

In practice, as shown in FIGS. 5 to 7, one or more cervical vertebrae 100 are the subject of an ablation of the central zone of the laminae, thereby individualizing, on a vertebra, two portions of lamina 101 with resected end surfaces 102.

One or more grafts 103, having lengths such that their ends can be situated, after placement of the implant 1, in the immediate vicinity of the resected surfaces 102, are placed through the conduit of the cage 3, then the implant 1 is inserted toward its implantation side on the vertebra 100 (see FIG. 5). As will be understood, this insertion is done in a cranial-caudal direction, i.e. parallel to the general longitudinal direction of the vertebral column, and continues until engagement of the inner side walls 15 on the inner side surfaces of the portions of lamina 101 and with the intermediate walls 17 bearing against the upper edges of said portions of lamina 101 (see FIG. 6). In that position, the ends of the graft(s) come into intimate contact with the resected surfaces 102.

The intermediate walls 17 are then deformed so as to fold the outer side walls 16 down against the outer surfaces of the portions of lamina 101 (see FIG. 7), and, in so doing, to insert the teeth of the side walls into the bone of the portions of lamina 101. Fastening screws (not shown) can then be placed through the holes comprised by said walls.

The implant 1 according to the invention thus comprises laminar anchoring side portions 6 whereof the engagement on the portions of lamina 101 is done in the cranial-caudal direction, using a much safer process, in terms of risk of lesion of the spinal cord, than the engagement of an implant according to the prior art, done in the antero-posterior direction, i.e. perpendicular to the general longitudinal direction of the vertebral column. Furthermore and above all, the implant 1 makes it possible to leave the resected surfaces 102 free and therefore to place one or more grafts 103 extending from one portion of lamina 101 to the other up to the vicinity of these resected surfaces 102. The reconstruction of a bone bridge between these portions of lamina 101 can then be done under the best possible conditions, by bone cell growth of the portions of lamina 101 toward the graft(s) 103. This bone bridge makes it possible to perform perfect reinsertion of the soft tissues normally anchored on a lamina or on a spinous process of a vertebra 100.

In the implantation position, the intermediate walls 17 bear against the upper edges of the portions of lamina 101 and thus form stop and stabilization surfaces of the implant 1.

The placement of the implant 1 is made easier and safer by spacing the outer side walls 16 apart from the inner walls 15; once they are folded down against the outer surfaces of the portions of lamina, said outer walls 16, jointly with the inner walls 16, make it possible to ensure complete fixing of the implant 1 to the portions of lamina 101.

The second embodiment of the implant 1, shown in FIGS. 8 and 9, is very similar to the first embodiment described above; it differs from the latter only in that the cage 3 forms a body with the base piece 2. Said cage 3 is therefore made from the same metallic material as the base piece 2.

The invention was described above in reference to embodiments provided as examples. It of course is not limited to those embodiments and extends to all other embodiments covered by the appended claims.

1-9. (canceled)

10. Laminoplasty implant in particular for cervical laminoplasty, adapted to connect the two portions of the lamina of a vertebra resulting from an ablation of the central zone of said lamina, comprising:
a body having a first end intended to be connected to a first portion of lamina and a second end intended to be connected to the second portion of lamina, said body having, in its implantation position, an outer side, an inner side, an upper side and a lower side; a first direction extends from the outer side toward the inner side of said body; and
two anchoring portions to said portions of lamina, one of which is connected to said first end and the other of which is connected to said second end, each anchoring portion being in the shape of a U, i.e. comprising an inner side wall intended to come against an inner side of the corresponding portion of lamina, an outer side wall intended to come against an outer side of said portion of lamina, and an intermediate wall connecting said inner side wall to said outer side wall; a second direction (d2) extends from said intermediate wall, substantially perpendicularly thereto, toward the opening of the U defined between the free end edges of said inner side wall and said outer side wall;
wherein
said intermediate wall of each anchor portion is situated toward the upper side of the body of the implant, and said second direction is substantially perpendicular to said first direction, said inner, outer and intermediate side walls thus being arranged relative to said body so that, when the body is arranged in said implantation position, the direction of engagement of these walls on the portions of lamina is parallel to the cranial-caudal direction of the vertebral column, i.e. the general longitudinal direction of said vertebral column, and
said central body comprises mounting means allowing one or more grafts to be mounted thereon that can extend from one resected surface of a portion of lamina to the resected surface of the other portion of lamina, so that the ends of that or those grafts can be situated, after placement, in the immediate vicinity of said resected surfaces.

11. Laminoplasty implant according to claim 10, characterized in that each intermediate wall is deformable between a first position, in which the outer side wall to which said intermediate wall is connected is remote from the inner side wall to which said intermediate wall is connected, and a second position, in which said outer side wall is closer to said inner side wall so as to grip a portion of lamina between it and said inner side wall.

12. Laminoplasty implant according to claim 10, characterized in that each inner side wall extends in a direction forming, with the inner side of the body of the implant, an angle going from 100 to 120°.

13. Laminoplasty implant according to claim 12, characterized in that each inner side wall extends in a direction forming, with the inner side of the body of the implant, an angle of 110°.

14. Laminoplasty implant according to claim 10, characterized in that each side wall and/or each intermediate wall comprise means favouring its fastening relative to a portion of lamina.

15. Laminoplasty implant according to claim 14, characterized in that said means favouring the fastening relative to a portion of lamina are sharp teeth that are able to be inserted into the bone of said portion of lamina, and/or one or more holes making it possible to place screws through said portion of lamina.

16. Laminoplasty implant according to claim 10, characterized in that said mounting means comprised by said central body for assembling one or more grafts are in the form of a cage having a transverse conduit, the ends of which emerge laterally.

17. Laminoplasty implant according to claim 16, characterized in that the cage assumes a flat triangular shape.

18. Laminoplasty implant according to claim 17, characterized in that the cage has concave lateral edges and rounded corners, and, seen in a transverse direction, a height that gets smaller on the side opposite said body.
